(19) United States
(12) Patent Application Publication
(10) Pub. No.: US 2023/0181577 A1
(43) Pub. Date: Jun. 15, 2023

(54) STABLE COMPOSITIONS OF VARENICLINE

(71) Applicant: MANKIND PHARMA LTD., New Delhi (IN)

(72) Inventors: Naveen Kumar MAURYA, Manesar (IN); Jayanthy Venkata Vijaya Narasimha KISHAN, Manesar (IN); Vivek JAIN, Manesar (IN); Chithambaram MUTHULINGAM, Manesar (IN); Amit BANSAL, Manesar (IN); Anil KUMAR, Manesar (IN)

(21) Appl. No.: 18/063,460

(22) Filed: Dec. 8, 2022

(30) Foreign Application Priority Data
Dec. 10, 2021 (IN) 202111057531

(51) Int. Cl. A61K 31/4985; A61K 9/20

(52) U.S. Cl. CPC A61K 31/4985; A61K 9/2095; A61K 9/2013; A61K 9/2054; A61K 9/2059

(57) ABSTRACT

The present invention relates to a pharmaceutical composition comprising Varenicline or its pharmaceutically acceptable salt with reduced amount of nitrosamine impurity and a process for preparing the same.

### FIELD OF INVENTION

The present invention relates to a stable pharmaceutical composition comprising Varenicline or its pharmaceutically acceptable salt with reduced amount of nitrosamine impurity and a process for preparing the same.

### BACKGROUND OF THE INVENTION

Varenicline is a partial nicotinic agonist selective for a43₂ nicotinic acetylcholine receptor subtypes. Varenicline is also known as 7,8,9,10-tetrahydro-6,10-methano-6H-pyrazino[2,3-h] [3]-benzazepine. and is represented by chemical Formula (I).

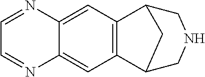 [SMILES: c1cnc2cc3c(cc2n1)C1CNCC3C1]

Varenicline, as the tartrate salt is indicated for use as an aid to smoking cessation treatment. It is available under the trade name of CHANTIX® tablets supplied in 0.5 mg and 1 mg strengths for oral administration.

U.S. Pat. No. 6,410,550 discloses Varenicline and its pharmaceutically acceptable acid addition salts.

US Pat. No. 6,890,927 discloses Varenicline Tartrate and process of its preparation.

US Pat. No. 7,265,119 discloses anhydrous L-tartrate salt of Varenicline and its process of preparation.

PCT application No. WO2006072832 discloses fast disintegrating dosage form of Varenicline Tartrate prepared by using co-processed carbohydrate.

PCT application No. WO2007122510 discloses osmotic composition of Varenicline or its pharmaceutically acceptable salt comprising a core containing at least one pharmaceutically active ingredient and at least one asymmetric membrane coating, wherein said coating comprises: a. one or more substantially water-insoluble polymers, and b. one or more solid, water-soluble polymeric materials selected from the group consisting of water-soluble cellulose derivatives, acacia, dextrin, guar gum, maltodextrin, sodium alginate, starch, polyacrylates, polyvinyl alcohols, and zein.

PCT application No. WO2010143070A2 discloses amorphous co-precipitate comprising varenicline tartrate and a pharmaceutically acceptable excipient selected from the group consisting of maltodextrin, lactose monohydrate, and 2-hydroxypropyl-β-cyclodextrin prepared by dissolving varenicline free base in an organic solvent and adding this to a solution of tartaric acid in water to obtain a solution, to the solution was added a solution of maltodextrin in water followed by spray drying using a spray dryer under high temperature condition.

Varenicline has been recalled from US market many times because of the presence of the nitrosamine impurity N-nitroso-varenicline at or above the FDA acceptable intake limit. There are multiple reasons why nitrosamines can be present in drugs. FDA found that the source of nitrosamines can be related to the drug's manufacturing process or its chemical structure or even the conditions in which they are stored or packaged. As foods and drugs are processed in the body, nitrosamines can also be formed. During process of preparing varenicline it is possible that trace amounts of nitrosating agents derived from prior nitration processes in nitro-substituted intermediates react subsequently with amines or substituted amines to form the corresponding N-substituted-N-nitrosamines. Nitrosamine impurity may increase the risk of cancer if people are exposed to them above acceptable levels and over longer periods of time.

There is always a need in art to develop a pharmaceutical composition of Varenicline or its pharmaceutically acceptable salt which is stable and the amount of nitrosamine impurity does not exceed beyond the FDA acceptable intake limit on long term storage.

### SUMMARY OF THE INVENTION

The present invention relates to a stable pharmaceutical composition comprising therapeutically effective amount of Varenicline or its pharmaceutically acceptable salt, one or more stabilizing agents, and one or more pharmaceutically acceptable excipients.

Another embodiment of the present invention provides a stable amorphous solid dispersion comprising Varenicline or its pharmaceutically acceptable salt, one or more stabilizing agents, and one or more pharmaceutically acceptable excipients.

Another embodiment of the present invention provides a stable amorphous solid dispersion comprising Varenicline or its pharmaceutically acceptable salt, one or more stabilizing agents selected from the group comprising maltodextrin, isomalt, meglumine, povidone, ascorbic acid, tocopherol, 2,5-dihydroxy benzoic acid, butylated hydroxytoluene, butylated hydroxyanisole, propyl gallates, or pullulan, and one or more pharmaceutically acceptable excipients.

The present invention further relates to a process for preparation of a stable pharmaceutical composition comprising Varenicline or its pharmaceutically acceptable salt, wherein the process comprises: (a) preparing a suspension or solution of Varenicline or its pharmaceutically acceptable salt, one or more stabilizing agents, and one or more pharmaceutically acceptable excipients in a solvent; (b) adding the suspension or solution of step (a) onto one or more pharmaceutical acceptable excipients to make granules; (c) compressing the granules to form tablets or filling the granules in capsules.

### DESCRIPTION OF THE INVENTION

The present invention relates to a stable pharmaceutical composition comprising therapeutically effective amount of Varenicline or its pharmaceutically acceptable salt, one or more stabilizing agents, and one or more pharmaceutically acceptable excipients.

As used in this specification, the singular forms “a”, “an”, and “the” include plural references unless the context clearly dictates otherwise. Thus for example, a reference to “a method” or “a process” includes one or more methods, one or more processes and/or steps of the type described herein and/or which will become apparent to those persons skilled in the art upon reading this disclosure and so forth.

The term “pharmaceutical composition” or “composition” or “solid oral composition” or “dosage form” or as used herein synonymously include tablet such as mono-layered tablets, bilayered tablets, trilayered tablet, multilayer tablet, caplets, minitablets, capsules, tablet in tablet, tablets in a capsule, granules in a capsule, pellets, pellets in a capsule, powder, granules, suspension or any other suitable dosage form meant for oral administration.

The terms “stable” according to present invention refers to a composition in which the amount of nitrosamine impurity remains below FDA acceptable intake limit after exposure to 40° C./75% RH for a period of six months or 25° C./60% RH. for a period of at least 12 months.

The terms “FDA acceptable intake limit” of nitrosamine impurity (N-nitroso-varenicline) as used in the present invention is the upper limit set by FDA for daily intake of nitrosamine impurity below which there is no harmful side-effect likely to occur on long term treatment with Varenicline or its pharmaceutically acceptable salt. The limit set by FDA for nitrosamine impurity in Varenicline drug products is 37 nanograms and below the interim acceptable intake limit of 185 ng of N-nitroso-varenicline per day. In accordance with the present invention, the Nitroso Varenicline is reduced to less than about 50 ppm, preferably less than about 25 ppm, more preferably less than about 5 ppm.

The term “pharmaceutically acceptable salt” or “salts” as used in the context of the present invention refers to inorganic acids such as hydrochloric acid, hydrobromic acid, sulphuric acid, phosphoric acid salt, carbonate salts; organic acids such as succinic acid, formic acids, acetic acid, diphenyl acetic acid, palmoic acid, triphenylacetic acid, caprylic acid, dichloroacetic acid, trifluoro acetic acid, propionic acid, butyric acid, lactic acid, citric acid, gluconic acid, mandelic acid, tartaric acid, malic acid, ascorbic acid, adipic acid, aspartic acid, fumaric acid, glutamic acid, maleic acid, malonic acid, benzoic acid, p-chlorobenzoic acid, dibenzoyl tartaric acid, oxalic acid, nicotinic acid, o-hydroxybenzoic acid, p-hydroxybenzoic acid, 1-hydroxy-naphthalene-2-carboxylic acid, hydroxynaphthalene carboxylic acid, ethanesulfonic acid, ethane-1,2-disulfonic acid, 2-hydroxyethane sulfonic acid, methanesulfonic acid, (+)-camphor-10-sulfonic acid, benzenesulfonic acid, naphthalene-2-sulfonic acid, p-toluenesulfonic acid and the like. The inorganic salts may further includes alkali metal and alkaline earth metal salts such as sodium, potassium, barium, lithium, calcium, magnesium, rhodium, zinc, cesium, selenium, and the like or, benethamine, benzathine, diethanolamine, ethanolamine, 4-(2-hydroxy-ethyl)morpholine, 1-(2-hydroxyethyl)pyrrolidine, N-methyl glucamine, piperazine, triethanol amine or tromethamine and the like.

The term “stabilizing agent(s)” as used in present invention is any suitable pharmaceutically acceptable excipient capable of keeping the limit of nitrosamine impurity below the FDA acceptable intake limit and is selected from the group comprising of antioxidants, amino acids, sugar alcohols, carbohydrates, pH modifiers, or a mixture thereof.

Antioxidant is selected from but not limited to, ascorbic acid, sodium ascorbate, alpha-tocopherol, butylated hydroxytoluene, butylated hydroxyanisole, caffeic acid, propyl gallate, ferulic acid, sodium pyrosulfite, edetic acid, edetate salts and the like.

Amino acid is selected from but not limited to glycine, lysine, histidine, and the like. Sugar alcohol is selected from mannitol, meglumine, isomalt, sorbitol, and the like. Carbohydrate is selected from but not limited to dextrins, maltodextrin, pullulan, cellulose derivatives, and the like.

pH modifier is selected from an acid selected from ascorbic acid, fumaric acid, citric acid, malic acid, and the like or a base selected from sodium carbonate, calcium carbonate, magnesium carbonate, sodium citrate, and the like

According to other embodiment, the stable pharmaceutical composition comprising Varenicline or its pharmaceutically acceptable salt, stabilizing agent, and one or more pharmaceutically acceptable excipients, wherein the nitrosamine impurity is less than about 50 ppm, preferably less than about 25 ppm, more preferably less than about 5 ppm.

According to one of the embodiments, Varenicline or its pharmaceutically acceptable salt and stabilizing agent(s) are present in the composition in a ratio of 1:1 to 1:25.

Another embodiment of the present invention provides a stable pharmaceutical composition comprising Varenicline or its pharmaceutically acceptable salt, antioxidant, and one or more pharmaceutically acceptable excipients.

According to one of the embodiments, Varenicline or its pharmaceutically acceptable salt and antioxidant(s) are present in the composition in a ratio of 1:1 to 1:25, preferably 1:1 to 1:10.

Another embodiment of the present invention provides a stable pharmaceutical composition comprising Varenicline or its pharmaceutically acceptable salt, maltodextrin, and one or more pharmaceutically acceptable excipients.

Another embodiment of the present invention provides a stable pharmaceutical composition comprising Varenicline or its pharmaceutically acceptable salt, ascorbic acid, and one or more pharmaceutically acceptable excipients.

Another embodiment of the present invention provides a stable pharmaceutical composition comprising Varenicline or its pharmaceutically acceptable salt, butylated hydroxytoluene, optionally butylated hydroxyanisole, and one or more pharmaceutically acceptable excipients. Another embodiment of the present invention provides a stable pharmaceutical composition comprising Varenicline or its pharmaceutically acceptable salt, butylated hydroxyanisole, and one or more pharmaceutically acceptable excipients.

Another embodiment of the present invention provides a stable pharmaceutical composition comprising Varenicline or its pharmaceutically acceptable salt, butylated hydroxyanisole, and one or more pharmaceutically acceptable excipients; wherein the ratio of Varenicline or its pharmaceutically acceptable salt to butylated hydroxyanisole is 1:1 to 1:10.

Another embodiment of the present invention provides a stable pharmaceutical composition comprising Varenicline or its pharmaceutically acceptable salt, pullulan, and one or more pharmaceutically acceptable excipients.

The term “excipient(s)” or “pharmaceutically acceptable excipient(s)” means a component of a pharmaceutical product that is not an active ingredient, and includes but not limited to fillers, diluents, disintegrants, glidants, lubricants, surface active agents etc. The excipients that are useful in preparing a pharmaceutical composition are generally safe, non-toxic, and neither biologically nor otherwise undesirable, and are acceptable for veterinary use as well as human pharmaceutical use. One excipient can perform more than one function.

Suitable fillers/diluents include, without limitation, starch, corn starch, potato starch, pregelatinized starch, dry starch, disaccharides, lactose, cellulose, cellulose derivatives, such as silicified microcrystalline cellulose, microcrystalline cellulose (e.g., cellulose MK GR), mannitol, sorbitol, xylitol, trehalose, colloidal silica, sucrose or other sugars or sugar derivatives such as maltodextrin, pullulan, calcium hydrogen phosphate, dicalcium phosphate, low-substituted hydroxypropyl cellulose, hydroxyethyl cellulose, hydroxypropyl methyl cellulose, and combinations thereof. When present, a filler may be employed in an amount ranging from about 10% to about 80%, preferably from about 20% to about 80% by weight of the pharmaceutical composition.

Suitable binders include, without limitation, microcrystalline cellulose, polyvinylpyrrolidone (PVP), e.g., PVP K 30 or PVP9OF, polyethylene glycols (PEG), e.g., PEG 4000, hydroxypropylmethyl cellulose, hydroxypropyl cellulose, both preferably of medium to high viscosity, e.g. viscosity grades 3 or 6 cps, copovidone, sugars and sugar alcohols, maltodextrin, sodium carboxymethylcellulose, gums, pregelatinized starch and combinations thereof. When present, a binder may be employed in an amount ranging from about 0.1% to about 20%, by weight of the pharmaceutical composition.

Suitable lubricants include, without limitation, zinc stearate, magnesium stearate, sodium stearyl fumarate, calcium stearate, stearic acid, colloidal silica, sodium lauryl sulfate, aluminum or calcium silicate, stearic acid, cutina, PEG 4000-8000, talc and combinations thereof. When present, a lubricant may be employed in an amount ranging from about 0.01% to about 10%, preferably from about 0.1% to about 5%, by weight of the pharmaceutical composition,

Suitable glidants include, without limitation, colloidal silicon dioxide (e.g., Aerosil 200), magnesium trisilicate, powdered cellulose, starch, talc and combinations thereof. When present, a glidant may be employed in an amount ranging from about 0.01% to about 10%, preferably from about 0.1% to about 5%, by weight of the pharmaceutical composition.

Suitable disintegrants include, without limitation, carboxymethylcellulose calcium (CMC-Ca), carboxymethylcellulose sodium (CMC-Na), crosslinked PVP (e.g. crospovidone, polyplasdone XL or kollidon CL), croscarmellose sodium, sodium starch glycolate, polacrillin potassium, low substituted hydroxypropyl cellulose, alginic acid, sodium alginate and guar gum, most preferably crosslinked PVP (crospovidone), crosslinked CMC (Ac-Di-Sol), carboxymethyl starch-Na (pirimojel and explotab) and/or combinations thereof. A disintegrant is employed in an amount of 0.01 to 15%, such as of 0.05 to 12%, such as at least 0.1 to 10%, by weight of the pharmaceutical composition.

The surfactants include but are not limited to anionic, cationic, non-ionic or amphoteric surfactants or those known to the person skilled in the art. Suitable surface active are poloxamer 188, polysorbate 80, Cremophore, Soluplus, lecithin and sodium lauryl sulfate.

Suitable coating agent according to present invention may selected from but not limited to, Shellac, cellulose acetate phthalate (CAP), polyvinylacetate phthalate (PVAP), hyroxylpropyl cellulose, hyroxypropyl methylcellulose (HPMC), and Zein (a corn protein derivative), Hydroxyproply methyl cellulose, Methyl hydroxyethyl cellulose, Ethylcellulose, Povidone, Opadry, and the like.

Another embodiment of the present invention provides a stable pharmaceutical composition comprising about 0.5 mg Varenicline free base or equivalent amount of pharmaceutically acceptable salt, about 2 mg of butylated hydroxyanisole, about 10 mg croscarmellose sodium, about 9 mg of maltodextrin, about 45 mg of microcrystalline cellulose, about 30 mg of dibasic calcium phosphate, about 0.5mg of colloidal silicon dioxide, about 1 mg of magnesium stearate.

Another embodiment of the present invention provides a stable pharmaceutical composition comprising about 1 mg Varenicline free base or equivalent amount of pharmaceutically acceptable salt, about 4 mg of butylated hydroxyanisole, about 20 mg croscarmellose sodium, about 18 mg of maltodextrin, about 90 mg of microcrystalline cellulose, about 60 mg of dibasic calcium phosphate, about 1 mg of colloidal silicon dioxide, about 2 mg of magnesium stearate.

Another embodiment of the present invention provides a stable amorphous solid dispersion of Varenicline or its pharmaceutically acceptable salt, one or more stabilizing agents, and one or more pharmaceutically acceptable excipients.

“Solid dispersion” as used herein refers to the dispersion of Varenicline or its pharmaceutically acceptable salt, where the active ingredient exists in solubilized or amorphous state. The increase in solubility of the drug in solid dispersion is mainly because drug remains in amorphous form which is associated with a higher energy state as compared to crystalline counterpart and due to that it requires very less external energy to dissolve. In order to arrive at such a solid dispersion, it is required to have a substantial amount of API dissolved in a suitable solvent at least at one-time point during preparation of the pharmaceutical composition.

Another embodiment of the present invention provides a stable amorphous solid dispersion comprising Varenicline or its pharmaceutically acceptable salt, one or more stabilizing agent selected from the group comprising maltodextrin, isomalt, meglumine, hydroxyethyl cellulose, povidone, ascorbic acid, tocopherol, 2,5-dihydroxy benzoic acid, butylated hydroxytoluene, butylated hydroxylanisole, propyl gallates, or pullulan, and one or more pharmaceutically acceptable excipients.

Another embodiment of the present invention provides a stable amorphous solid dispersion comprising Varenicline or its pharmaceutically acceptable salt, one or more antioxidants, and one or more pharmaceutically acceptable excipients.

Another embodiment of the present invention provides a stable amorphous solid dispersion comprising Varenicline or its pharmaceutically acceptable salt, maltodextrin, and one or more pharmaceutically acceptable excipients.

Another embodiment of the present invention provides a stable amorphous solid dispersion comprising Varenicline or its pharmaceutically acceptable salt, ascorbic acid, and one or more pharmaceutically acceptable excipients.

Another embodiment of the present invention provides a stable amorphous solid dispersion comprising Varenicline or its pharmaceutically acceptable salt, butylated hydroxytoluene, optionally butylated hydroxyanisole, and one or more pharmaceutically acceptable excipients.

Another embodiment of the present invention provides a stable amorphous solid dispersion comprising Varenicline or its pharmaceutically acceptable salt, butylated hydroxyanisole, and one or more pharmaceutically acceptable excipients.

Another embodiment of the present invention provides a stable amorphous solid dispersion comprising Varenicline or its pharmaceutically acceptable salt, butylated hydroxyanisole, and one or more pharmaceutically acceptable excipients; wherein the ratio of Varenicline or its pharmaceutically acceptable salt to butylated hydroxyanisole is 1:1 to 1:10.

Another embodiment of the present invention provides a stable amorphous solid dispersion comprising Varenicline or its pharmaceutically acceptable salt, pullulan, and one or more pharmaceutically acceptable excipients.

The present invention further relates to a process for preparation of stable pharmaceutical composition comprising Varenicline or its pharmaceutically acceptable salt, wherein the process comprises: (a) preparing a suspension or solution of Varenicline or its pharmaceutically acceptable salt, one or more stabilizing agents, and one or more pharmaceutically acceptable excipients in a solvent; (b) adding the suspension or solution of step (a) onto one or more pharmaceutical acceptable excipients to make granules; (c) compressing the granules to form tablets or filling the granules in capsules.

Suitable solvent used for preparing solid dispersion of Varenicline or its pharmaceutically acceptable salt is selected from, but not limited to, the group comprising of alcohol such as methanol, ethanol, 2-nitroethanol, 2-fluoroethanol, 2,2,2-trifluoroethanol, hexafluoroisopropyl alcohol, ethylene glycol, 1-propanol, 2-propanol (isopropyl alcohol), 2-methoxyethanol, 1-butanol, 2-butanol, t-butyl alcohol, 2-ethoxyethanol, diethylene glycol, polyethylene glycol, 1-, 2-, or 3-pentanol, neo-pentyl alcohol, t-pentyl alcohol, diethylene glycol monomethyl ether, diethylene glycol monoethyl ether, cyclohexanol, phenol, glycerol and the like; halogenated solvent such as dichloromethane, chlorobenzene, tetrachloromethane, 1,2-dichloroethane, trichloroethylene, perchloroethylene, 1,1,1-trichloroethane, 1,1,2-trichloroethane, chloroform and the like; ketone such as acetone, methyl ethyl ketone, methyl isobutyl ketone, diethyl ketone, methyl t-butyl ketone and the like; ester solvent such as methyl acetate, ethyl acetate, isopropyl acetate, n-propyl acetate, propenyl acetate, t-butyl acetate, isobutyl acetate, n-butyl acetate, ethyl formate, methyl acetate, methyl propanoate, ethyl propanoate, methyl butanoate, ethyl butanoate and the like; hydrocarbon solvent such as toluene, xylene, heptane, cyclohexane and the like, ether such as tetrahydrofuran, methyl tetrahydrofuran, dioxane, diethyl ether, diisopropyl ether, methyl t-butyl ether, glyme, diglyme, dibutyl ether, dimethylfuran, 2-methoxyethanol, 2-ethoxyethanol, anisole and the like; nitrile such as acetonitrile, propionitrile, butanenitrile and the like; water; and a combination thereof.

According to one embodiment the process for preparation of a stable pharmaceutical composition comprises:
a) preparing a solution of Varenicline tartrate in water followed by addition of maltodextrin to the solution,
b) preparing a solution of butylated hydroxyanisole in isopropyl alcohol,
c) granulating a blend of excipients selected form the group consisting of diluent selected from one or more of cellulose derivatives, sugars and sugar alcohols, microcrystalline cellulose, mannitol, sorbitol, xylitol, trehalose, sucrose, calcium hydrogen phosphate, dicalcium phosphate, or a combination thereof; disintegrant is carboxymethylcellulose calcium, carboxymethylcellulose sodium, croscarmellose sodium, sodium starch glycolate, polacrillin potassium, low substituted hydroxypropyl cellulose, crosslinked polyvinyl pyrrolidine, carboxymethyl starch sodium; lubricant selected from one or more of zinc stearate, magnesium stearate, sodium stearyl fumarate, calcium stearate, stearic acid, colloidal silica, sodium lauryl sulfate, aluminium silicate, calcium silicate, stearic acid, polyethylene glycol; binders selected microcrystalline cellulose, polyvinylpyrrolidone (PVP), polyethylene glycols (PEG), hydroxypropylmethyl cellulose, hydroxypropyl cellulose, copovidone, sugars and sugar alcohols, maltodextrin, sodium carboxymethylcellulose, gums, pregelatinized starch with the solution of step (a) and (b), to make granules,
d) drying the granules, lubricating, and compressing the granules to obtain tablets.

According to one embodiment the process for preparation of a stable pharmaceutical composition comprises:
a) preparing a solution of Varenicline tartrate in water followed by addition of maltodextrin to the solution,
b) preparing a solution of butylated hydroxyanisole in isopropyl alcohol,
c) granulating a blend of microcrystalline cellulose, dicalcium phosphate, and croscarmellose, to make granules, and
d) drying the granules, lubricating, and compressing the granules to obtain tablets.

Another embodiment of the present invention provides a process for preparation of a stable pharmaceutical composition, wherein the pharmaceutical composition has less than about 50 ppm nitrosamine impurity after exposure of the pharmaceutical composition to 40° C./75% RH for a period of six months, and less than about 500 ppm of adduct impurity of Formula A:

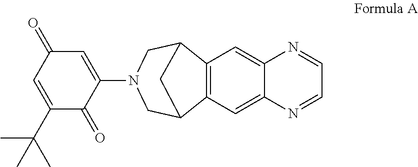 [SMILES: CC(C)(C)C1=CC(=O)C=C(N2CC3CC(C2)c2cc4nccnc4cc23)C1=O]

after exposure of the pharmaceutical composition to 40° C./75% RH for a period of six months, preferably wherein the pharmaceutical composition has less than about 25 ppm nitrosamine impurity after exposure of the pharmaceutical composition to 40° C./75% RH for a period of six months, and less than about 250 ppm of adduct impurity of Formula A after exposure of the pharmaceutical composition to 40° C./75% RH for a period of six months.

According to other embodiment, varenicline is spray dried with one or more stabilizing agents. The rapid evaporation of spray drying achieves yet another advantage, which is the formation of particles having relatively uniform particle size distribution and shape. The particles formed by rapid evaporation have better flow characteristics and are less likely to become segregated during manufacturing, such as during handling to form tablets. This is particularly important for a drug such as varenicline, since the drug itself has a high potency and therefore is used at a low dose. Reducing segregation during manufacture of the dosage form is important to ensure uniformity of dose in the dosage form. Thus, spray-drying reduces segregation during manufacturing of the dosage form by providing varenicline in a form that is easier to handle. Once the varenicline-containing spray dried composition has been formed, several processing operations can be used to facilitate incorporation of the varenicline into a dosage form. These processing operations include drying, granulation, milling, compression, and the like.

The pharmaceutical composition of present invention can be formulated by any suitable granulation methods known in the art such as wet granulation, direct compression, dry granulation, or melt granulation. More specifically, wet granulation process using rapid mixer granulator.

According to one embodiment the process for preparation of a stable pharmaceutical composition comprises:
i) preparing a solution of butylated hydroxyanisole in Isopropyl alcohol, and
ii) preparing a solution of Varenicline Tartrate in water followed by addition of Maltodextrin to obtain a drug binder solution;
iii) blending croscarmellose sodium, microcrystalline cellulose, dibasic calcium phosphate followed by granulating the blend with the solution of step i) and step ii) in a rapid mixer granulator to obtain granules,
iv) drying the granules followed by milling to obtain dried and milled granules;
v) lubricating the dried and milled granules followed by compressing to obtain tablets, and
vi) optionally coating the tablets.

The present invention provides an adduct impurity represented by Formula A,

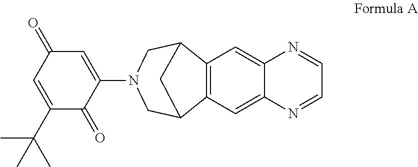 [SMILES: CC(C)(C)C1=CC(=O)C=C(N2CC3CC(C2)c2cc4nccnc4cc23)C1=O]

The adduct impurity of Formula A is used as reference standard for characterization of impurities in varenicline or its pharmaceutically acceptable salt.

According to other embodiment, the stable pharmaceutical composition comprising Varenicline or its pharmaceutically acceptable salt, stabilizing agent, and one or more pharmaceutically acceptable excipients having reduced to no-detectable amount of an adduct impurity represented by Formula A,

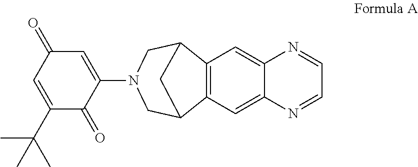 [SMILES: CC(C)(C)C1=CC(=O)C=C(N2CC3CC(C2)c2cc4nccnc4cc23)C1=O]

wherein the adduct impurity is less than about 500 pm, preferably less than about 250 ppm.

Another embodiment of the present invention provides a stable pharmaceutical composition comprising:
0.5% w/w-1.0% w/w of Varenicline or its pharmaceutically acceptable salt;
1% w/w—20% w/w of stabilizing agent selected from the group consisting of isomalt, meglumine, povidone, ascorbic acid, tocopherol, 2,5-dihydroxy benzoic acid, butylated hydroxytoluene, butylated hydroxyanisole, propyl gallates, pullulan, or a combination thereof;
10% w/w—80% w/w of the diluent selected from the group consisting of cellulose derivatives, microcrystalline cellulose, sugars and sugar alcohols, mannitol, sorbitol, xylitol, trehalose, sucrose, maltodextrin, pullulan, calcium hydrogen phosphate, dicalcium phosphate, or a combination thereof,
1% w/w—10% w/w of disintegrant selected from the group consisting of carboxymethylcellulose calcium, carboxymethylcellulose sodium, croscarmellose sodium, sodium starch glycolate, polacrillin potassium, low substituted hydroxypropyl cellulose, crosslinked polyvinyl pyrrolidine, carboxymethyl starch sodium, or a combination thereof;
1% w/w—10% w/w of the binder selected from the group consisting of microcrystalline cellulose, polyvinylpyrrolidone (PVP), polyethylene glycol (PEG), hydroxypropylmethyl cellulose, hydroxypropyl cellulose, copovidone, sugars and sugar alcohols, maltodextrin, sodium carboxymethylcellulose, gums, pregelatinized starch, or a combination thereof, or a combination thereof; and
0.1% w/w—5% w/w of lubricant selected from the group consisting of zinc stearate, magnesium stearate, sodium stearyl fumarate, calcium stearate, stearic acid, colloidal silica, sodium lauryl sulfate, aluminium silicate, calcium silicate, stearic acid, polyethylene glycol or a combination thereof; based on the total weight of the composition.

Another embodiment of the present invention provides a stable pharmaceutical composition of Varenicline or its pharmaceutically acceptable salt, wherein:
the stabilizing agent is butylated hydroxytoluene, butylated hydroxyanisole, or a combination thereof;
the diluent is microcrystalline cellulose, dicalcium phosphate, or a combination thereof;
the disintegrant is croscarmellose sodium, crosslinked polyvinyl pyrrolidine or a combination thereof;
the binder is hydroxypropylmethyl cellulose, maltodextrin, polyvinylpyrrolidone; and
the lubricant is magnesium stearate, colloidal silicon dioxide, or combination thereof, wherein the amount of nitrosamine impurity after exposure of the pharmaceutical composition to 40° C./75% RH for a period of six months is less than about 25 ppm and adduct impurity is less than about 250 ppm.

The present invention is further illustrated by the following examples which are provided merely to be exemplary of the invention and don't limit the scope of the invention. Certain modifications and equivalents will be apparent to those skilled in the art and are intended to be included within the scope of the present invention.

### EXAMPLE 1

### Pharmaceutical Composition of Varenicline with Maltodextrin

| Ingredient | Quantity % w/w |
|---|---|
| Varenicline Tartrate | 0.814 |
| Maltodextrin | 8.143 |
| Microcrystalline Cellulose | 46.75 |
| Anhydrous dibasic calcium phosphate | 28.579 |
| Croscarmellose sodium | 9.524 |
| Colloidal Silicon Dioxide | 0.476 |
| Magnesium stearate | 0.952 |
| Opadry coat | 4.762 |

Process:
(a) Microcrystalline cellulose, anhydrous dibasic calcium phosphate, and other ingredients were dispensed and sifted.
(b) Maltodextrin was dissolved in purified water under continuous stirring. Varenicline Tartrate was added to the solution of maltodextrin in water and dissolved under continuous stirring to form solution of Varenicline Tartrate with maltodextrin in water.
(c) Blend of step a) was granulated using the solution of step b) followed by blending the granules together with Croscarmellose sodium, Microcrystalline cellulose and Colloidal silicon dioxide.
(d) Blend of step (c) was lubricated with Magnesium stearate.
(e) Lubricated blend of step (d) was compressed using suitable punches and dies to form tablets.
(f) Tablets of step (e) were coated with coating solution.

### EXAMPLE 2

### Pharmaceutical Composition of Varenicline with Pullulan

| Ingredient | Quantity % w/w |
|---|---|
| Varenicline Tartrate | 0.814 |
| Pullulan | 8.143 |
| Anhydrous dibasic calcium phosphate | 75.805 |
| Croscarmellose sodium | 9.524 |
| Magnesium stearate | 0.952 |
| Opadry coat | 4.762 |

Process for preparation of composition of Example 2 is similar to the process followed in Example 1 except solution of Varenicline Tartrate with pullulan in water was prepared.

### EXAMPLE 3

### Pharmaceutical Composition of Varenicline with Meglumine and Hydroxypropyl Cellulose

| Ingredient | Quantity % w/w |
|---|---|
| Varenicline Tartrate | 0.814 |
| Hydroxypropyl cellulose | 8.143 |
| Meglumine | 5.700 |
| Microcrystalline Cellulose | 41.057 |
| Anhydrous dibasic calcium phosphate | 28.572 |
| Croscarmellose sodium | 9.524 |
| Colloidal Silicon Dioxide | 0.476 |
| Magnesium stearate | 0.952 |
| Opadry coat | 4.762 |

Process for preparation of composition of Example 3 is similar to the process followed in Example 1 except Varenicline tartrate was added to the solution of Hydroxypropyl cellulose and Meglumine.

### EXAMPLE 4

### Pharmaceutical Composition of Varenicline with Ascorbic Acid and Hydroxypropyl Cellulose

| Ingredient | Quantity % w/w |
|---|---|
| Varenicline Tartrate | 0.814 |
| Hydroxypropyl cellulose | 8.143 |
| Ascorbic acid | 4.286 |
| Microcrystalline Cellulose | 42.46 |
| Anhydrous dibasic calcium phosphate | 28.59 |
| Croscarmellose sodium | 9.52 |
| Colloidal Silicon Dioxide | 0.473 |
| Magnesium stearate | 0.952 |
| Opadry coat | 4.762 |

Process for preparation of composition of Example 3 is similar to the process followed in Example 1 except Varenicline tartrate was added to the solution of Hydroxypropyl cellulose and Ascorbic acid.

### Example 5

### Pharmaceutical Composition of Varenicline with Ascorbic Acid and Maltodextrin

| Ingredient | Quantity % w/w |
|---|---|
| Varenicline Tartrate | 0.814 |
| Maltodextrin | 8.143 |
| Ascorbic acid | 4.071 |
| Microcrystalline Cellulose | 42.68 |
| Anhydrous dibasic calcium phosphate | 28.57 |
| Croscarmellose sodium | 9.52 |
| Colloidal Silicon Dioxide | 0.478 |
| Magnesium stearate | 0.962 |
| Opadry coat | 4.762 |

Process:
(a) Croscarmellose sodium & microcrystalline cellulose and other ingredients were dispensed and sifted.
(b) Maltodextrin and ascorbic were dissolved in purified water under continuous stirring. Varenicline Tartrate was added to the solution of maltodextrin in water and dissolved under continuous stirring to form solution of Varenicline Tartrate with maltodextrin in water.
(c) Blend of step (a) was granulated using solution of step b) and c) followed by blending with croscarmellose sodium, microcrystalline cellulose, and colloidal silicon dioxide.
(d) Blend of step (d) was lubricated using magnesium stearate.
(e) Lubricated blend of step (e) was compressed using suitable punches and dies to form tablets.
(f) Tablets of step (e) were coated with coating solution.

### Example 6

### Pharmaceutical Composition of Varenicline with Butylated Hydroxytoluene, Butylated hydroxyanisole and Maltodextrin

| Ingredient | Quantity % w/w |
|---|---|
| Varenicline Tartrate | 0.814 |
| Maltodextrin | 8.143 |
| Butylated hydroxytoluene | 0.357 |
| Butylated hydroxyanisole | 1.904 |
| Microcrystalline Cellulose | 73.80 |
| Croscarmellose sodium | 9.32 |
| Stearic acid | 0.90 |
| Opadry Pink 20A540023 | 4.762 |

Process:
(a) Croscarmellose sodium & microcrystalline cellulose and other ingredients were dispensed and sifted.
(b) Solution of butylated hydroxyanisole and butylated hydroxytoluene was prepared using isopropyl alcohol under continuous stirring.
(c) Maltodextrin was dissolved in purified water under continuous stirring. Varenicline Tartrate was added to the solution of maltodextrin in water and dissolved under continuous stirring to form solution of Varenicline Tartrate with maltodextrin in water.
(d) Blend of step (a) was granulated using solution of step b) and c) followed by blending with croscarmellosesodium & microcrystalline cellulose.
(e) Blend of step (d) was lubricated using stearic acid.
(f) Lubricated blend of step (e) was compressed using suitable punches and dies to form tablets.
(g) Tablets of step (f) were coated with coating solution.

### Example 7

### Pharmaceutical Composition of Varenicline with Butylated Hydroxyanisole

| | | | | |
|---|---|---|---|---|
| S. No | Name of Ingredients | 0.5 mg | 1 mg | % w/w |

Drug Binder Solution

| | | | | |
|---|---|---|---|---|
| 1 | Varenicline Tartrate | 0.855 | 1.710 | 0.814 |
| 2 | Maltodextrin | 8.550 | 17.100 | 8.143 |
| 3 | Purified water | Q.s. | Q.s. | |

Butylated Hydroxyanisole (BHA) Solution

| | | | | |
|---|---|---|---|---|
| 4 | Butylated Hydroxyanisole | 2.000 | 4.000 | 1.905 |
| 5 | Isopropyl Alcohol | Q.s. | Q.s. | |

Dry Mixing

| | | | | |
|---|---|---|---|---|
| 6 | Microcrystalline Cellulose | 42.095 | 84.190 | 40.090 |
| 7 | Anhydrous Dibasic Calcium | 30.000 | 60.000 | 28.571 |
| | Phosphate | | | |
| 8 | Croscarmellose Sodium | 5.000 | 10.000 | 4.762 |

Pre-lubrication

| | | | | |
|---|---|---|---|---|
| 9 | Microcrystalline Cellulose | 5.000 | 10.000 | 4.762 |
| 10 | Colloidal Silicon Dioxide | 0.500 | 1.000 | 0.476 |
| 11 | Croscarmellose Sodium | 5.000 | 10.000 | 4.762 |

Lubrication

| | | | | |
|---|---|---|---|---|
| 12 | Magnesium Stearate | 1.000 | 2.000 | 0.952 |

| | | |
|---|---|---|
| Average weight of | 100.000 | 200.000 |
| core tablets (mg) | | |

Film coating

| | | | | |
|---|---|---|---|---|
| 13 | Opadry White 20A58900 | 5.000 | — | |
| 14 | Opadry pink 20A540023 | — | 10.000 | 4.762 |
| 15 | Purified Water^{#} | Q.s. | Q.s. | |

| | | |
|---|---|---|
| Average weight of | 105.000 | 210.000 |
| coated tablets (mg) | | |

Process:

1. Sifting: Croscarmellose sodium & microcrystalline cellulose and other ingredients were dispensed and sifted.

2. BHA solution preparation: Isopropyl alcohol was taken in a suitable vessel & Butylated Hydroxyanisole was slowly added under continuous stirring.

3. Drug Binder Solution preparation: Varenicline Tartrate was added to purified water and dissolved under continuous stirring. Maltodextrin was added to step under continuous stirring.

4. Blending: Croscarmellose sodium & Microcrystalline cellulose, Anhydrous Dibasic Calcium phosphate were loaded in Rapid Mixer Granulator (RMG) and blended for suitable time.

5. Granulation: Blend of step 4 was granulated with a solution of step 2 & 3. for suitable time in rapid mixer granulator (RMG).

6. Drying: Granules of step 5 were dried in a fluid bed dryer (FBD) at 45° C.±10° C. to achieve suitable LOD.

7. Sifting/Milling: Dried granules of step 5 were sifted through a suitable sieve and retains/oversize granules were milled in a suitable mill to get uniform sized dried granules.

8. Blending: Granules obtained from step 7 were loaded in a suitable blender and blended for suitable time with prelubrication blend of Croscarmellose sodium, microcrystalline cellulose, and colloidal silicon dioxide using suitable blender for suitable time.

9. Lubrication: Blend from step 8 was lubricated with Magnesium stearate in a suitable blender for suitable time.

10. Compression: Lubricated blend of step 9 was compressed using rotary compression machine with suitable punches and dies for respective strength.

11. Coating: Opadry was dispersed in purified water under continuous stirring for suitable time. Dispersion was sifted. Tablets were coated with the dispersion.

Stability Studies: The pharmaceutical compositions of the Example 7 (0.5 mg and 1 mg) were tested for stability at 25° C./60% RH and 40° C./75% RH for a period of six months. The amount of nitrosamine impurity is provided in table 1 below.

TABLE 1

| Test | | | | 25° C./60% RH | | 40° C./75% RH | |
|---|---|---|---|---|---|---|---|
| Parameters | Specification | Batch No. | Initial | 3 M | 6 M | 3 M | 6 M |

0.5 mg

| | | | | | | | |
|---|---|---|---|---|---|---|---|
| Varenicline N | NMT 1.0% | FNJKV001 | BQL | BQL | 0.03 | 0.07 | 0.13 |
| formyl Impurity | | | | | | | |
| Varenicline | NMT 0.5% | | BQL | BQL | BQL | 0.07 | 0.10 |
| Adduct impurity | | | | | | | |
| at RRT 3.2 | | | | | | | |
| Any unspecified | NMT 0.5% | | 0.12 | 0.14 | 0.13 | 0.06 | 0.12 |
| degradation | | | | | | | |
| product | | | | | | | |
| Total degradation | NMT 2.5% | | 0.27 | 0.28 | 0.21 | 0.38 | 0.61 |
| products | | | | | | | |
| Nitrosamine | NMT 18.5 | | BDL | BDL | BDL | BDL | BDL |
| impurity | ppm | | | | | | |

1 mg

| | | | | | | | |
|---|---|---|---|---|---|---|---|
| Varenicline N | NMT 1.0% | FWJKV001 | BQL | BQL | BQL | 0.07 | 0.12 |
| formyl Impurity | | | | | | | |
| Varenicline | NMT 0.5% | | BQL | BQL | BQL | 0.13 | 0.18 |
| Adduct impurity | | | | | | | |
| at RRT 3.2 | | | | | | | |
| Any unspecified | NMT 0.5% | | 0.11 | 0.14 | 0.12 | 0.07 | 0.05 |
| degradation | | | | | | | |
| product | | | | | | | |
| Total degradation | NMT2.5% | | 0.15 | 0.14 | 0.12 | 0.43 | 0.47 |
| products | | | | | | | |
| Nitrosamine | NMT 18.5 | | BDL | BDL | BDL | BDL | BDL |
| impurity | ppm | | | | | | |

BDL is Below Detection Limit of Nitrosamine impurity: 0.92 parts per million
BQL is Below Quantification Limit of Nitrosamine impurity: 2.78 parts per million

### Example 8

### Preparation of Stable Solid Dispersion of Varenicline Tartrate with Maltodextrin

A mixture of Varenicline Tartrate (0.5 g) and maltodextrin (0.5 g) was dissolved in water (25 mL) at 25° C. and filtered the solution to make it particle free. The solvent was evaporated in rotavapour under reduced pressure at 50° C. to solid dispersion of Varenicline Tartrate.

1. A stable pharmaceutical composition comprising therapeutically effective amount of Varenicline or its pharmaceutically acceptable salt, one or more stabilizing agents, and one or more pharmaceutically acceptable excipients selected for the group comprising diluent, binder, disintegrant, lubricant, or glidant.

2. The stable pharmaceutical composition according to claim 1, wherein the amount of nitrosamine impurity after exposure of the pharmaceutical composition to 40° C./75% RH for a period of six months is less than about 50 ppm.

3. The stable pharmaceutical composition according to claim 1, wherein the ratio of Varenicline or its pharmaceutically acceptable salt to the stabilizing agent is 1:1 to 1:25.

4. The stable pharmaceutical composition according to claim 1, wherein the stabilizing agent is selected from the group consisting of isomalt, meglumine, povidone, ascorbic acid, tocopherol, 2,5-dihydroxy benzoic acid, butylated hydroxytoluene, butylated hydroxyanisole, propyl gallates, pullulan, or a combination thereof.

5. The stable pharmaceutical composition according to claim 4, wherein the stabilizing agent is butylated hydroxyanisole.

6. The stable pharmaceutical composition according to claim 5, wherein the ratio of Varenicline or its pharmaceutically acceptable salt to butylated hydroxyanisole is 1:1 to 1:10.

7. The stable pharmaceutical composition according to claim 5, wherein the pharmaceutical composition has less than about 500 ppm of adduct impurity of formula A: 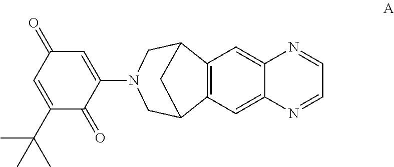 [SMILES: CC(C)(C)C1=CC(=O)C=C(N2CC3CC(C2)c2cc4nccnc4cc23)C1=O]
after exposure of the pharmaceutical composition to 40° C./75% RH for a period of six months.

8. The stable pharmaceutical composition according to claim 5, wherein the amount of nitrosamine impurity is less than about 25 ppm and the amount of adduct impurity of formula A: 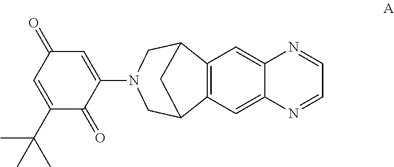 [SMILES: CC(C)(C)C1=CC(=O)C=C(N2CC3CC(C2)c2cc4nccnc4cc23)C1=O]
is less than about 250 ppm, after exposure of the pharmaceutical composition to 40° C./75% RH for a period of six months.

9. The stable pharmaceutical composition according to claim 1, comprising:
0.5% w/w—1.0% w/w of Varenicline or its pharmaceutically acceptable salt;
1% w/w—20% w/w of the stabilizing agent selected from the group consisting of isomalt, meglumine, povidone, ascorbic acid, tocopherol, 2,5-dihydroxy benzoic acid, butylated hydroxytoluene, butylated hydroxyanisole, propyl gallates, pullulan, or a combination thereof;
10% w/w—80% w/w of the diluent selected from the group consisting of cellulose derivatives, microcrystalline cellulose, sugars and sugar alcohols, mannitol, sorbitol, xylitol, trehalose, sucrose, maltodextrin, pullulan, calcium hydrogen phosphate, dicalcium phosphate, or a combination thereof,
1% w/w—10% w/w of the disintegrant selected from the group consisting of carboxymethylcellulose calcium, carboxymethylcellulose sodium, croscarmellose sodium, sodium starch glycolate, polacrillin potassium, low substituted hydroxypropyl cellulose, crosslinked polyvinyl pyrrolidine, carboxymethyl starch sodium, or a combination thereof;
1% w/w—10% w/w of the binder selected from the group consisting of microcrystalline cellulose, polyvinylpyrrolidone (PVP), polyethylene glycol (PEG), hydroxypropylmethyl cellulose, hydroxypropyl cellulose, copovidone, sugars and sugar alcohols, maltodextrin, sodium carboxymethylcellulose, gums, pregelatinized starch, or a combination thereof, or a combination thereof; and
0.1% w/w—5% w/w of the lubricant selected from the group consisting of zinc stearate, magnesium stearate, sodium stearyl fumarate, calcium stearate, stearic acid, colloidal silica, sodium lauryl sulfate, aluminium silicate, calcium silicate, stearic acid, polyethylene glycol or a combination thereof; based on the total weight of the composition.

10. The stable pharmaceutical composition according to claim 9, wherein
the stabilizing agent is butylated hydroxytoluene, butylated hydroxyanisole, or a combination thereof;
the diluent is microcrystalline cellulose, dicalcium phosphate, or a combination thereof;
the disintegrant is croscarmellose sodium, crosslinked polyvinyl pyrrolidine or a combination thereof;
the binder is hydroxypropylmethyl cellulose, maltodextrin, polyvinylpyrrolidone; and
the lubricant is magnesium stearate, colloidal silicon dioxide, or combination thereof, wherein the amount of nitrosamine impurity is less than about 25 ppm and adduct impurity is less than about 250 ppm after exposure of the pharmaceutical composition to 40° C./75% RH for a period of six months.

11. The stable pharmaceutical composition according to claim 10, comprising about 0.5 mg Varenicline free base or equivalent amount of pharmaceutically acceptable salt, about 2 mg of butylated hydroxyanisole, about 10 mg croscarmellose sodium, about 9 mg of maltodextrin, about 45 mg of microcrystalline cellulose, about 30 mg of dibasic calcium phosphate, about 0.5mg of colloidal silicon dioxide, about 1 mg of magnesium stearate.

12. The stable pharmaceutical composition according to claim 10, comprising about 1 mg Varenicline free base or equivalent amount of pharmaceutically acceptable salt, about 4 mg of butylated hydroxyanisole, about 20 mg croscarmellose sodium, about 18 mg of maltodextrin, about 90 mg of microcrystalline cellulose, about 60 mg of dibasic calcium phosphate, about 1 mg of colloidal silicon dioxide, about 2 mg of magnesium stearate.

13. A compound of formula A,

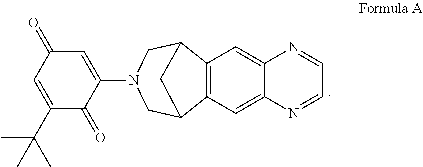 [SMILES: CC(C)(C)C1=CC(=O)C=C(N2CC3CC(C2)c2cc4nccnc4cc23)C1=O]

14. A process for preparation of a stable pharmaceutical composition comprising Varenicline or its pharmaceutically acceptable salt, wherein the process comprises: (a) preparing suspension(s) or solution(s) of Varenicline or its pharmaceutically acceptable salt, one or more stabilizing agents, and optionally one or more pharmaceutically acceptable excipients in a solvent together or separately; (b) adding the suspension(s) or solution(s) of step (a) onto one or more pharmaceutical acceptable excipients to make granules; (c) compressing the granules to form tablets or filling the granules in capsules.

15. The process for preparation of a stable pharmaceutical composition according to claim 14, wherein step (a) comprises preparing a solution of Varenicline tartrate and maltodextrin in water and preparing a solution of butylated hydroxyanisole in isopropyl alcohol and step (b) comprises granulating a blend of microcrystalline cellulose, dicalcium phosphate, and croscarmellose sodium with the solution of Varenicline tartrate and maltodextrin in water and butylated hydroxyanisole in isopropyl alcohol, to make granules.

16. The process for preparation of a stable pharmaceutical composition according to claim 15, wherein the step (b) comprises granulating the blend in a rapid mixer granulator.

17. The process for preparation of a stable pharmaceutical composition according to claim 14,
wherein step (a) comprises:
i) preparing a solution of butylated hydroxyanisole in Isopropyl alcohol, and
ii) preparing a solution of Varenicline Tartrate in water followed by addition of Maltodextrin to obtain a drug binder solution;
wherein step (b) comprises:
i) blending croscarmellose sodium, microcrystalline cellulose, dibasic calcium phosphate followed by granulating the blend with the solution of step i) and step ii) in a rapid mixer granulator to obtain granules,
ii) drying the granules followed by milling to obtain dried and milled granules;
c) lubricating the dried and milled granules followed by compressing to obtain tablets, and
d) optionally coating the tablets.

18. The process for preparation of a stable pharmaceutical composition according to claim 14, wherein the pharmaceutical composition has less than about 50 ppm of nitrosamine impurity after exposure of the pharmaceutical composition to 40° C./75% RH for a period of six months and less than about 500 ppm of adduct impurity of Formula A: 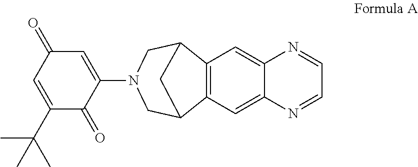 [SMILES: CC(C)(C)C1=CC(=O)C=C(N2CC3CC(C2)c2cc4nccnc4cc23)C1=O]
after exposure of the pharmaceutical composition to 40° C./75% RH for a period of six months.

19. The process for preparation of a stable pharmaceutical composition according to claim 17, wherein the pharmaceutical composition has less than about 25 ppm of nitrosamine impurity after exposure of the pharmaceutical composition to 40° C./75% RH for a period of six months, and less than about 250 ppm of adduct impurity of Formula A: 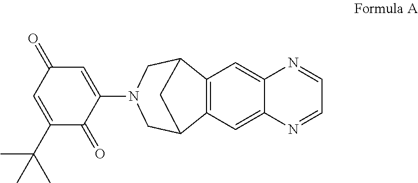 [SMILES: CC(C)(C)C1=CC(=O)C=C(N2CC3CC(C2)c2cc4nccnc4cc23)C1=O]
after exposure of the pharmaceutical composition to 40° C./75% RH for a period of six months.

20. A stable pharmaceutical composition prepared by process according to claim 17.
